(19) United States
(12) Patent Application Publication
(10) Pub. No.: US 2007/0088352 A1
(43) Pub. Date: Apr. 19, 2007

(54) Method and system for radio frequency ophthalmological presbyopia surgery

(76) Inventor: Robert S. Rosen, Solana Beach, CA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/250,351

(22) Filed: Oct. 14, 2005

(51) Int. Cl. A61B 18/18

(52) U.S. Cl. 606/45; 606/41

(57) ABSTRACT

An ophthalmological surgical method and system for treating presbyopia in a living is provided. The method and system entail applying radio frequency energy to the sclera of the eye to produce a pattern of grooves therein. The pattern includes at least one furrow having a width in the range of approximately 200 to approximately 2000 microns and a depth in the range of approximately ninety percent to slightly less than one hundred percent of the thickness of the sclera.

### CROSS REFERENCE TO RELATED APPLICATIONS

“Not Applicable”

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### BACKGROUND OF THE INVENTION

1. Field if the Invention

This invention relates generally to ophthalmological surgery and more particularly to surgical methods for treating presbyopia by applying radio frequency energy to the sclera.

2. Description of Related Art

With aging, a condition of the eye known as presbyopia develops. With this condition, the crystalline lens of the eye loses the ability to focus on near objects. Presbyopia is often treated with bifocal eyeglasses. With bifocals, one portion of the lens corrects for abnormalities of far-vision (e.g., myopia or hyperopia), and another portion of the lens corrects for near-vision. Efforts have been made to treat presbyopia using partitioned contact lenses positioned directly over the pupil of the eye. Examples include multifocal contact lenses. Unfortunately, when presbyopia is corrected with bifocal or multifocal lenses attached to the cornea, the user is simultaneously looking through the near and far vision-correcting lenses. As a result, the user will see both in-focus and out-of-focus images simultaneously when viewing an object. This out-of-focus image superimposed on the in-focus image can cause glare and degrade vision when viewing objects at low contrast.

Another technique for treating presbyopia is to correct one eye of the subject for near-vision and to correct the other eye for far-vision. This technique is known as monovision. With monovision, a subject uses one eye to see distant objects and the other eye to see near objects. Unfortunately, with monovision, the subject may not clearly see objects that are intermediately positioned because the object is out-of-focus for both eyes. Monovision may result in loss of depth perception.

Methods for treating a presbyopic subject have been proposed and are found in the patent literature. For example, in U.S. Pat. No. 5,354,331 (Schachar) there is disclosed a method for treating presbyopia and hyperopia. The method ostensibly increases the amplitude of accommodation by increasing the effective working distance of the ciliary muscle in the presbyopic eye, e.g., the effective working distance of the ciliary muscle can be increased by expanding the sclera in the region of the ciliary body by suturing to the sclera in the region of the ciliary body a relatively rigid band having a diameter slightly greater than that of the sclera in that region. The scleral expansion band comprises anterior and posterior rims and a web extending between the rims, the anterior rim having a smaller diameter than the posterior rim. Other methods for increasing the diameter of the sclera in the region of the ciliary body may also be used. For example, the sclera may be thinned or weakened by the surgical removal of a portion of its collagenous substance, as, for example by paring or by abrading the surface or by ablating the surface with laser irradiation.

In U.S. Pat. No. 5,489,299 (Schachar) there is disclosed a method for treating presbyopia and hyperopia are treated by a method which increases the amplitude of accommodation by increasing the effective working distance of the ciliary muscle in the presbyopic eye. The effective working distance of the ciliary muscle can be increased by expanding the sclera in the region of the ciliary body. This patent discloses that the expansion can be accomplished by suturing to the sclera a relatively rigid band having a diameter slightly greater than that of the sclera in the region of the ciliary body, by weakening the sclera overlying the ciliary body, by surgical procedures or treatment with enzymes, heat or radiation, whereby intraocular pressure expands the weakened sclera, or by surgical alloplasty. The effective working distance of the ciliary muscle can also be increased by shortening the zonules by application of heat or radiation, by repositioning one or both insertions of the ciliary muscle or by shortening the ciliary muscle.

In U.S. Pat. No. 6,745,775 (Lin) there is disclosed an ophthalmic surgery method for treating presbyopic patient by removing a portion of the scleral tissue of an eye in a predetermined pattern and area utilizing a laser, whereby the accommodation of the presbyopic eye increases via the movement of the ciliary body and zonular fiber connected to the lens of the eye. The predetermined pattern has a depth of about (60%-90%) of a scleral tissue thickness. The proposed laser wavelength ranges from ultraviolet to infrared of (0.15-0.36) microns, (0.5-1.4) microns and (0.9-10.6) microns. Stable accommodation is achieved by the filling of the sub-conjunctival tissue to the laser-ablated scleral areas. Both scanning and fiber delivered systems are proposed.

In U.S. Pat. No. 5,413,574 (Fugo) there is disclosed a method of ocular surgery wherein low power radio waves are transmitted from the tip of an active incising electrode and used to make incisions in the tissues of the eye. One disclosed technique is for treating glaucoma, wherein the radio waves are used to create full thickness holes for drainage. The method makes use of a high impedance contact between the surgical subject and a grounding plate connected to the radio wave generator. The use of the low power radio wave energy and the high impedance contact prevents the active incising electrode from becoming hot and causing damage to sensitive tissues of the eye.

In U.S. Pat. No. 5,423,815 (Fugo) there is disclosed a method of ocular refractive surgery which employs heat application to reshape and enhance the refractive power of the central cornea of a surgical subject. Radio frequency energy is employed to coagulate segments of corneal stroma in the perilimbal area at the pole of the corneal meridian having the lowest keratometric reading, causing the radius of curvature of the central cornea to increase in that meridian, whereby astigmatism can be safely and permanently corrected.

While the techniques and methods of the above mentioned patents may be suitable for their intended purposes a need nevertheless exists for a method of treating presbyopia in a living being that does not make use of laser to effect a weakening of the scleral tissue to enable the circumference of the eye to expand. The subject invention addresses that need.

### BRIEF SUMMARY OF THE INVENTION

In accordance with one aspect of this invention there is provided a ophthalmological surgical method and system for treating presbyopia in a living being.

The method basically entails applying radio frequency energy to the sclera of the eye in a desired pattern to remove portions of the scleral tissue in that pattern. The pattern basically comprises at least one furrow. The at least one furrow has a width in the range of approximately 200 to approximately 2000 microns, and a depth in the range of approximately ninety percent to slightly less than one hundred percent, e.g., ninety-nine percent, of the thickness of the sclera.

In one preferred aspect of the invention the pattern comprises plural furrows, which may be of the same shape and size or different shapes and/or sizes.

The at least one furrow is created using a radio frequency device, e.g., an electro-cautery device. That device has an electrode tip arranged for application to the sclera of the eye and another electrode for engagement with a portion of the body of the patient. The device is operated by bringing the tip adjacent the scleral tissue of the eye so that the electrode tip removes a portion of the scleral tissue in the desired pattern.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF SEVERAL VIEWS OF THE DRAWING

FIGS. 1A and 1B are illustrations of the respective right and left eyes of a patient showing the results of the method of the subject invention on those eyes, e.g., the formation of an exemplary pattern of furrows created in the sclera of each eye by use of radio-frequency energy to thereby weaken the sclera thereat and thus treat the patient's presbyopia;

FIGS. 2A is a plan view of one exemplary marker/template arranged for disposition centered over the eye of a patient to mark the sclera of that eye with indicia indicating the position of a desired pattern of furrows so that radio-frequency energy can be applied to the indicia to create the pattern of furrows;

FIGS. 2B is a plan view of another exemplary marker/template arranged for disposition centered over the eye of a patient for marking the eye with indicia to facilitate the surgical procedure of this invention;

FIG. 3 is an illustration of one eye of a patient that has been marked with indicia by use of either of the exemplary markers/templates of FIGS. 2A and 2B to designate the location of the pattern of furrows to be produced;

FIG. 4 is an illustration shows the eye of a patient that has been marked with one exemplary pattern, e.g., eight “hockey-stick” shaped lines, showing where the furrows will be produced to result in the exemplary treated eyes shown in FIGS. 1A and 1B.

FIGS. 5A-5E are five exemplary patterns for furrows (out of a myriad of different shaped furrows) which can be produced using the subject invention to treat presbyopia;

FIG. 6 is a cross-sectional view of a portion of the eye showing that the depth of an exemplary furrow in the sclera produced in accordance with the method of the subject invention;

FIG. 7 is an exemplary device, e.g., an electro-cautery device, for delivery radio-frequency energy to the eye to produce one or more furrows in the sclera in accordance with this invention; and

FIG. 8 is an illustration showing the use of the device of FIG. 7 to create each furrow.

### DETAILED DESCRIPTION OF THE PREFERRED EMBODIMENT

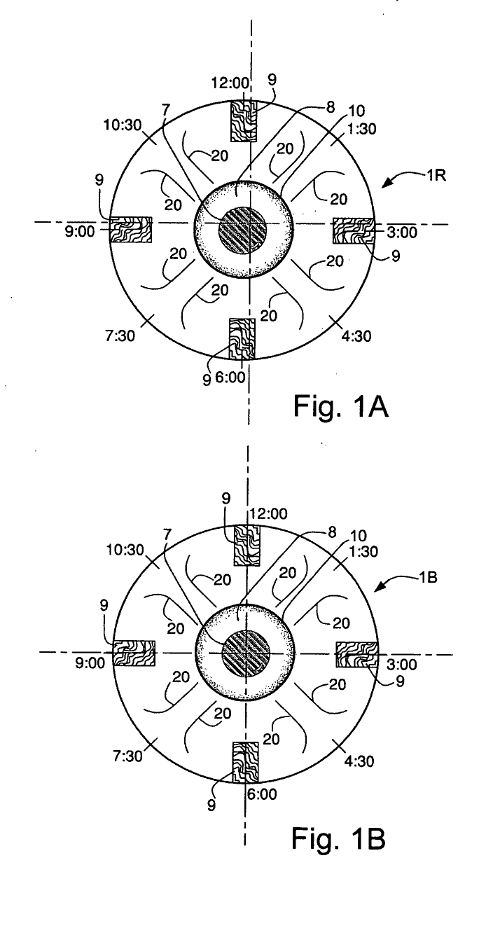

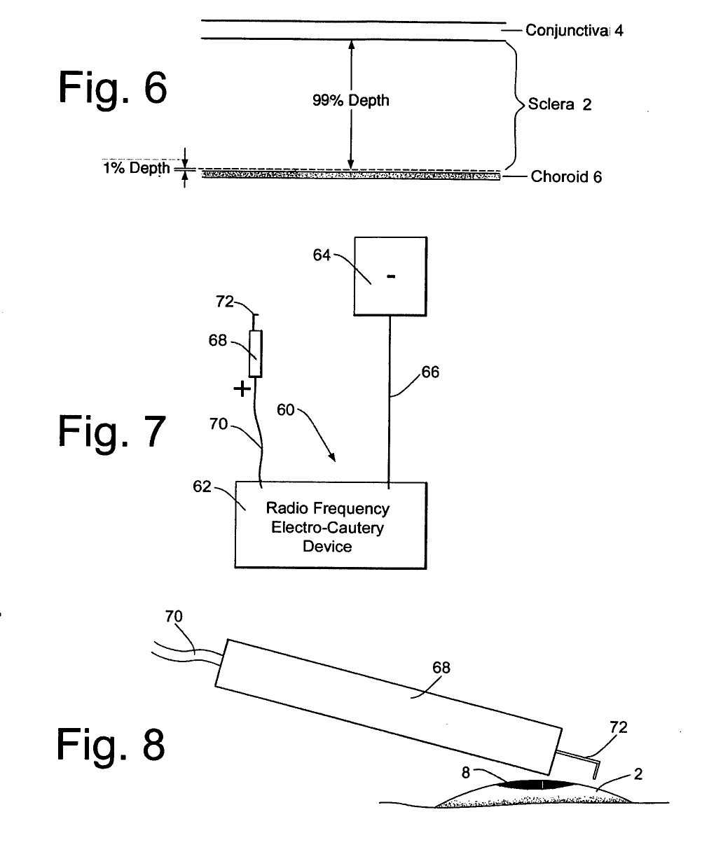

Referring now to the various figures of the drawing wherein like reference characters refer to like parts, there is shown in FIGS. 1A and 1B respective right and left eyes 1R and 1L of a patient which have been treated by the surgical procedure of this invention to correct or otherwise lessen that patient's presbyopia. The method of the subject invention centers around using radio-frequency ablation to create a pattern of one or more grooves or furrows 20 in the sclera 2, wherein the furrow depth is greater than ninety percent and just slightly less than one-hundred percent, e.g., is approximately ninety-nine percent, of the subject's scleral thickness. As used herein, “sclera” means the collagenous outer-wall of the eyeball comprising mostly collagen and some elastic tissue, which is covered by conjunctiva. A cross-sectional view of this portion of the eye is shown in FIG. 6. As can be seen in that figure the conjunctiva 4 is located over the sclera 2 and the choroid 6 is located under the sclera 2.

In accordance with this invention, the surgeon can prepare any number of furrows 20 in the sclera 2 depending on the subject's age, condition of the sclera, refractive error, and visual needs. Thus, a surgeon can prepare 1, 2, 3, 4, 5, 6, 7, 8, 9, 10, 11, 12, 13, 14, 15, 16 or more furrows in the sclera of an eye. In the exemplary embodiment shown in FIGS. 1A and 1B the surgeon has created two furrows 20 in each quadrant of sclera of an eye, thereby resulting in a pattern having a total of eight furrows. This is merely one example of a myriad of furrow patterns that can be produced in accordance with this invention.

It must be pointed out at this juncture that the surgical treatment of presbyopia in accordance with the subject invention does not require the formation of any particular pattern of furrows, nor does it require the use of multiple furrows or furrows of a particular shape. Thus, the number, arrangement and shape of the furrows, be it one or plural furrows of the same shape (such as shown in FIGS. 1A and 1B) or differing shapes), is left to the discretion of the surgeon to best treat that particular patient's presbyopia based on the patient's age, condition of the sclera, refractive error, visual needs and any other relevant factors.

Before going into the specifics of the subject invention it should be understood that the specific aspects of the invention described below are not limited to the specific administration methods and apparatus disclosed herein. Thus, the methods and apparatus for carrying out the invention may vary. It is also to be understood that the terminology used herein is for the purpose of describing particular aspects only and is not intended to be limiting. Moreover, as used in the specification and the appended claims, the singular forms “a,” “an” and “the” include plural referents unless the context clearly dictates otherwise. Thus, for example, reference to “a furrow” includes one, two or more furrows and the like. Further still, as used throughout, the term “patient” or “subject” or “living being” is meant to denote an individual. Moreover, that individual can include not only living human beings, but domesticated animals, such as cats, dogs, etc., livestock (e.g., cattle, horses, pigs, sheep, goats, etc.), wild animals, laboratory animals (e.g., mouse, rabbit, rat, guinea pig, etc.) and birds. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “approximately” or “about”, it will be understood that the particular value forms another aspect. It will be further understood that the endpoints of each of the ranges are significant both in relation to the other endpoint, and independently of the other endpoint.

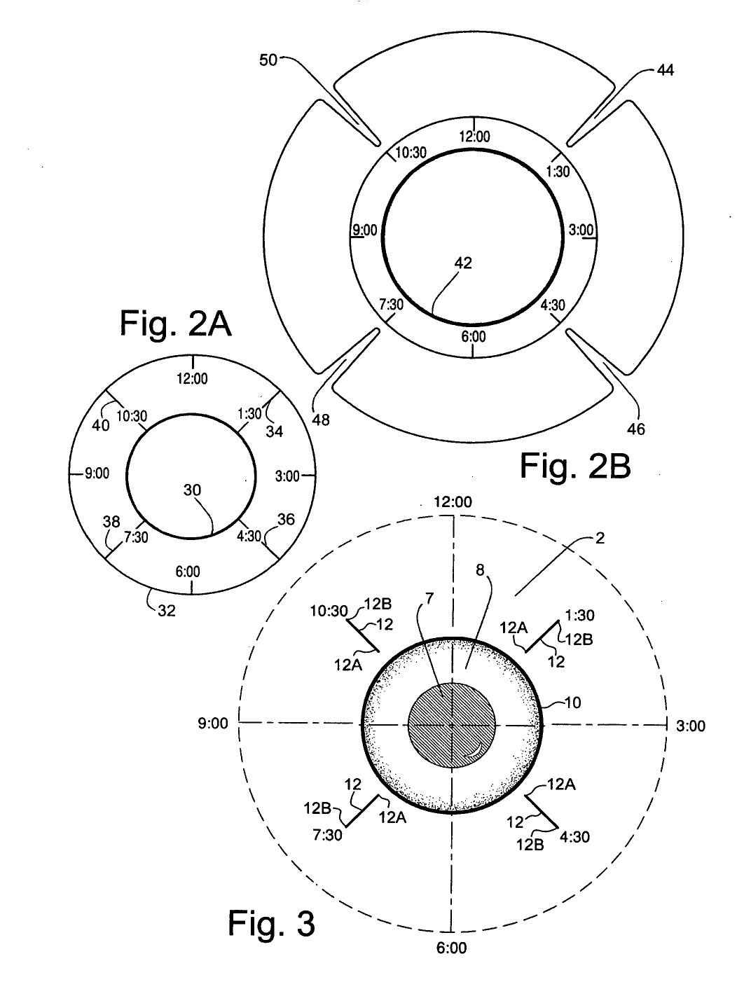

The method of the subject invention will be described in detail later. Suffice it for now to state that it basically comprises providing a template or marker, e.g., template/marker 22 (FIG. 2A) or template/marker 22′ (FIG. 2B) on the eye to be treated so that at least one visible indicium or mark can be placed on the eye to designate the location where the furrow(s) is/are to be produced. Once the indicium/indicia mark(s) have been placed on the eye, radio-frequency energy is applied to the sclera in the desired place(s) designed by that/those mark(s) and directed along a desired path to ablate most of the thickness of the sclera and thereby create the furrow(s) of the desired shape, size, position and orientation.

In order to carry out the method of this invention the patient should be kept comfortable, preferably with a minimal amount of locally administered anesthesia. In unusual situations, for example, when a subject is allergic to all agents used in local anesthesia, general anesthesia can be administered.

To decrease bleeding, a topical vasoconstrictive agent can be used such as Naphcone® A, Alphagan® P or Neosephrine® 2.5%-10%. Local anesthesia can be administered to a subject by one or more of several routes, for example, topically, by sub-conjunctival injection, by sub-Tenons injection, by peribulbar injection and by retrobulbar injection, or by other methods as would be clear to one skilled in the art. Anesthesia can be supplemented, if necessary, during the procedure to ensure that the subject is comfortable. Whether supplementation is necessary can be determined by the surgeon performing the described methods or by another of skill in the art using common diagnostic and clinical techniques. Examples of agents used topically (i.e., instilled onto the eyeball) include, but are not limited to, 0.5% to 1.0% Tetracaine® ophthalmic solution, 0.5% to 1.0% Proparacaine® ophthalmic solution, and 1.0% to 2.0% Lidocaine® gel. Examples of agents that can be administered by injection include, but are not limited to, 1.0% to 2.0% Lidocaine® and 0.75% Marcaine®, with or without epinephrine. To induce anesthesia of the cornea and conjunctiva, for example, 1.0% Proparacaine® can be instilled onto the surface of an eye approximately 5 minutes before the ablation procedure begins and can be given periodically during the course of the procedure at time intervals known in the art, for example every 5 minutes. Alternatively, topical 2.0% Lidocaine® gel can be applied to the surface of an eye 20-30 minutes prior to surgery. Still another method of inducing anesthesia is to apply a pledget saturated with 1.0% Tetracaine® ophthalmic solution around the circumference of the globe overlying the ciliary body for approximately 5 minutes before surgery. A subject can then be prepared and draped in a usual sterile ophthalmic manner. A lid speculum, for example, a wire lid speculum, can be placed around the eyeball to retract the upper and lower lids from the eyeball to allow the surgeon access to the surgical field. To achieve greater anesthesia, the surgeon can then administer one or more of the drugs listed above for injection subconjunctivally; into sub-Tenon's space; peribulbarly in one or more of the superonasal, inferonasal, inferotemporal and superotemporal quadrants of the eye; and/or retrobulbarly. For example, a peribulbar injection consisting of a 50%-50% mixture of 2.0% Lidocaine® and 0.75% Marcaine®, with or without epinephrine, can be administered according to the clinical judgment of the surgeon if the subject needs more anesthesia. After anesthesia is achieved and a conventional corneal protector (not shown) is placed on the eyeball, a 360 degree fornix-based conjunctival peritomy can be prepared. Hemostasis can be achieved by methods known in the art, e.g., using a conventional hand-held cautery device. Using blunt dissection with Wescott scissors and forceps, for example, a surgeon can dissect the conjunctiva and Tenon's capsule off the underlying sclera anterior to and between the insertions of the superior, medial, inferior and lateral rectos muscles. The scleral surface is cleaned from about the surgical limbus anteriorly to about eight millimeters posteriorly.

Two exemplary marker/templates 22 and 22′ that can be used for marking the eye with the indicium/indicia are shown in FIGS. 2A and 2B, respectively. The details of each of these marker/templates will be described later. Suffice it for now to state that each marker/template includes indicia in the form of radially extending lines and associated numbers indicating the 12:00, 1:30, 3:00, 4:30, 6:00, 7:30, 9:00 and 10:30 positions that are used to facilitate the marking of the eye with indicia serving as the pattern for the furrow(s) to be created.

As can be seen in FIG. 2A the indicia of the template/marker 22 at the 1:30, 4:30, 7:30 and 10:30 positions are generally longer that the indicia at the 12, 3, 6 and 9 o'clock positions. For example it is contemplated that the indicia the 1:30, 4:30, 7:30 and 10:30 positions can be approximately 2 mm in length, and the indicia at the 12, 3, 6 and 9 o'clock position can be approximately 1 mm, although other lengths can be used, as will be appreciated by one skilled in the art.

As shown in FIG. 1B, and as will be described later, the marker/template 22′ has notches at one or more of any of the disclosed positions, e.g., at the 1:30, 4:30, 7:30 and 10:30 positions. These notches serve as a stencil to allow a surgeon to place marks on the sclera in the region defined by the notches.

Either template/marker 22 or 22′ of FIGS. 2A or 2B, respectively, is temporarily placed on the ocular surface. In particular, the surgeon places the template/marker 22/22′ on the eye so that the pupil 7 and the iris 8 are located in the center of the template/marker. The template/marker is oriented so that each indicium can be visualized by the surgeon, with the 12, 3, 6 and 9 o'clock indicia aligned approximately with the lengths of the corresponding rectos muscles 9. Those muscles are shown schematically in FIGS. 1A and 1B. This alignment defines four quadrants on the surface of the eyeball as shown by dashed lines in FIGS. 1A and 1B. Thus, as shown in FIG. 1A, which is a right eye, the area from the 12 o'clock position to the 3 o'clock position forms the superonasal quadrant; the area from the 3 o'clock position to 6 o'clock position forms the inferonasal quadrant; the area from the 6 o'clock position to the 9 o'clock position forms the inferotemporal quadrant; and the area from the 9 o'clock position to the 12 o'clock position forms the superotemporal quadrant. As will be appreciated by one skilled in the art, exact alignment at the 12, 3, 6 and 9 o'clock positions is not necessary to practice the method of this invention, and as such, deviation from these positions is contemplated by this invention. For example, as the eyeball has a tendency to rotate, the template/marker 22 can be positioned at about 12, 3, 6 and 9 o'clock +/−10 degrees or at +/−5 degrees. Other template/marker positions can also be used.

Referring now to FIG. 2A, the details of that embodiment of the marker/template 22 will now be described. In particular, the template/marker 22 basically comprises a pair of concentric rings 30 and 32, which are interconnected by four radially extending arms 34, 36, 38 and 40 located between the rings 30 and 32 at the 1:30, 4:30, 7:30 and 10:30 positions. The marker/template 22 can be formed of any suitable material, e.g., plastic or metal and is an integral unit. The diameter of the inner ring 30 is slightly larger than diameter of the surgical limbus 10, so that when the marker/template 22 is placed centered over the eye the inner end of each of the arms 34, 36, 38 and 40 extends from a position located a short distance beyond, e.g., approximately 0.25 mm to 3 mm, the surgical limbus. These arms terminate at the outer ring 32. An ink, such as sterile ink used to mark skin for plastic surgery, or some other indicia-producing ophthalmic marking medium can be applied to the underside of the arms so that when the marker/template 22 is placed on the eye and centered, the ink is transferred to the contiguous portions of the conjunctiva 4 or sclera 2 to result in marking the eye with indicia like shown in FIG. 3. Those indicia are referred to hereinafter as “primary marks” 12 and in the exemplary embodiment shown are linear and radially extending. It should be noted that the marker/template 22 can be modified so that its arms 34, 36, 38 and 40 do not apply ink or some other marking medium to the conjunctiva, but rather depress or create visible indentations in the conjunctiva (and possibly the underlying sclera) by the surgeon pressing on the marker/template 22.

The marker/template 22′ of FIG. 2B basically comprises a member formed of any suitable material, e.g., plastic or metal and is shaped to generally conform to the outer periphery of the eye, e.g., it has an inner concave surface conforming to the shape of the eye and an outer convex surface of similar curvature. The center of the marker/template 22′ includes a central circular opening 42 or clear window through which the pupil and iris can be seen. Indicia, like the indicia on the marker/template 22, are provided at equidistantly spaced locations about the periphery of the central opening 42. Thus, these indicia are at the 12:00, 1:30, 3:00, 4:30, 6:00, 7:30, 9:00 and 10:30 positions. The outer periphery of the marker/template 22′ is generally circular and concentric with the central opening 42. Four narrow V-shaped notches 44, 46, 48 and 50 are located in outer periphery of the marker/template 22′ at the 1:30, 4:30, 7:30 and 10:30 positions and extend radially outward. The inner end of each of the notches terminates at a location a short distance beyond the surgical limbus 10. A marking pen or some other indicia producing device (not shown) can be extended through each of the notches 44, 46, 48 and 50 to apply ink or some other ophthalmic marking medium to the contiguous portions of the surface of the conjunctiva to result in marking the eye with the primary marks 12 like shown in FIG. 3. Alternatively, some instrument (not shown) can be inserted through the notches to result in the indentation of the conjunctiva (and possibly the underlying sclera) which are visible to the surgeon.

As shown in FIG. 3, at about the 1:30, 4:30, 7:30 and 10:30 position (meridians) of the eyeball, the primary marks 12 are located over the sclera 2 from about 0.25 mm to about 3 mm posterior to the surgical limbus 10 (the place on the surface of the eyeball near where the cornea meets the sclera and the iris can no longer be visualized through the cornea). Any conventional sterile ophthalmic marking ink or other marking media can be used. The primary marks 12 can be generally linear with a first end 12A located from about 0.25 mm to about 3 mm posterior to the surgical limbus and a second end 12B located posterior to the first end by approximately 2 mm to 7 mm. The marks 12 can also take the form of a dot, or a dotted or solid line, or another shape as would be clear to one skilled in the art. As described above, positions other than exactly at 1:30, 4:30, 7:30 and 10:30 can be used. For example, these positions +/−10 degrees or +/−5 degrees can be used. Moreover, marks can be made at other positions based on clinical judgment. The marker/template 22 and 22′ (FIGS. 2A and 2B, respectively) can be removed from the surgical field after the primary marks 12 are made leaving ink or pressure marks. Other suitable methods of marking the sclera, can also be used, for example, a small mark can be made using a hand-held cautery device.

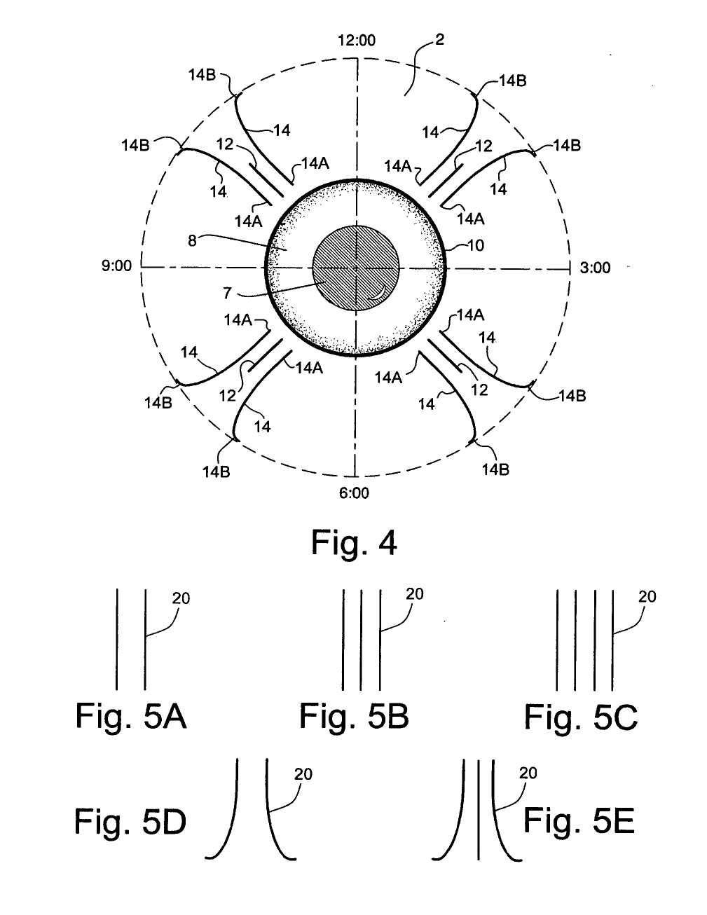

Once the primary marks 12 are provided on the eye, secondary marks to indicate the position, shape, size and orientation of the furrows to be produced are then placed on the eye at desired locations. For example, in the exemplary embodiment shown in FIG. 4, on both sides of each of the four primary marks 12, a pair of an secondary marks 14 are provided. These marks may be produced by means of a ophthalmic marking ink or some other marking medium. Moreover, while two secondary marks 14 are shown on each side of the primary marks 12, that is merely exemplary. Thus, only one secondary mark may be located adjacent a primary mark. In fact, the primary mark 12 can also serve as the location for secondary mark or may form the secondary mark itself. In the exemplary embodiment shown in FIG. 4 each secondary mark 14 includes a most anterior aspect 14A and a most posterior aspect 14B. The secondary marks can be placed on the eye at any desired distance, e.g., approximately 1 mm to 1.5 mm, from the primary mark, and with the most anterior aspect 14A approximately 0.75 mm posterior to the surgical limbus 10. Thus, in the embodiment shown eight secondary marks 14, two in each quadrant, are extended posteriorly in a radial manner. The posterior extent of each secondary mark 14 overlies the pars plana and can curve so that the most posterior aspect of the mark is nearly parallel to the limbus. The secondary marks 14 in each quadrant can be mirror images of each other. For example, two radial secondary marks in each quadrant can be separated from each other by from about 2 mm to about 3 mm. One particularly suitable spacing is about 2.5 mm. The most anterior aspect 14B of the two marks 14 can be separated by about 2 mm, and the posterior extent of those marks can be separated from each other by from about 2.5 mm to about 3 mm.

It must be pointed out at this juncture that the secondary marks 14 shown in FIG. 4 are exemplary of a myriad of shapes, sizes, orientations and spacing of patterns for furrows to be produced in accordance with this invention. To that end, in FIGS. 5A-5E there are shown five different shaped furrow patterns out of a myriad of possible furrow patterns that can be formed in one or more of the quadrants of the sclera.

Once the eye has been suitably marked with the desired pattern, it is ready for the surgeon to create the furrows corresponding to that pattern. To that end, a conventional corneal protector is placed over the cornea to prevent bright light from the operating microscope from damaging the retina. The surgeon then applies radio-frequency energy, by means of any suitable device, such as an electro-cautery device 60, like shown in FIG. 7, to the sclera 2 along one or more of each of the marks 14 to ablate the scleral strictures in one or more quadrants. For example, the surgeon apply the tip of the device 60 (to be described later) to two radial marks in each of the four quadrants to result in the furrow pattern shown in FIGS. 1A and 1B. As used herein “scleral strictures” refer to sites of sclerosis and rigidity (loss of elasticity) of the subject's sclera. Alternatively, the surgeon can make only one furrow 20 in each quadrant of the sclera along the course of either of the two secondary marks 14. (FIG. 4).

Each furrow 20 has a generally smooth floor that follows the curvature of the eyeball, and the walls of each furrow are approximately perpendicular to the floor. To create each furrow the surgeon holds the tip of the radio-frequency applying device 60 in contact with the conjunctiva or a short distance from the conjunctiva at the location of the mark(s) 14, such as shown in FIG. 8, and then moves the tip along the length of the mark in an antero-posterior and postero-anterior manner so that the tip ablates the underlying sclera.

Referring now to FIG. 7 the details of one particularly suitable device 60 for ablating the sclera with radio-frequency energy is shown. That device basically comprises a conventional electro-incision medical instrument for performing electrosurgery of the eye, such as sold by Medisurge Research and Management Corp. of Norristown, Pa., under the model designation Fugo Blade®. The device 60 basically comprises a power unit or generator 62, a relatively large area grounding plate 64 electrically connected to the generator by an electrical conductor or cable 66, and a hand-holdable electrode 68 electrically connected to the generator by an electrical conductor or cable 70. The hand-holdable electrode 68 includes a working tip 72, which constitutes the incising or ablating tip. The grounding plate 64 is arranged to be placed in contact with any suitable portion of the body of the patient. The generator 62 produces a low power radio-frequency energy, with the amount of energy produced being adjustable. To that end, when the device 60 is operated a voltage differential is produced between the electrode tip 72 and the grounding plate 64, e.g, the electrode tip is at some positive potential. The amount of power provided is adjustable. Electrode tips 72 of differing sizes can be used with the device 60 to create the desired size (e.g., width and depth) furrow(s). To that end the device 60 can be selectively provided with tips of various sizes, e.g., from about 50 to about 2000 microns in diameter. The low power radio wave energy generated by the generator is fed to the active incising tip 72. A high impedance exists between the patient and the grounding plate 64 so that the incising tip 72 is prevented from becoming hot by virtue of the high impedance between the patient and the return path via the grounding plate.

Operation of the device 60 to create any furrow 20 is as follows: The surgeon powers the device 60 and brings the tip 72 into contact with or closely adjacent to the portion of the eye bearing the secondary mark 14 and moves the tip therealong to create a furrow 20 that corresponds to that mark. The mark can be linear or curvilinear over the its length. Each furrow 20 can be from approximately 3 mm to approximately 7 mm in length and from approximately 200 microns to approximately 2000 microns in width. For example, the width of the furrow can be about 600 microns in width. A skilled surgeon can ablate greater than 90%, 91%, 92%, 93%, 94%, 95%, 96%, 97%, 98% or 99% of the sclera to form the floor and walls of each furrow, thereby removing at least 500 microns thickness of sclera in each furrow (FIG. 6). The subject's scleral thickness can be measured pre-operatively, and the ablation depth can be controlled accordingly. Periodic irrigation of the surgical field with a sterile ophthalmic balanced salt solution can prevent drying and thinning of the exposed sclera.

When a surgeon just begins to see a bluish color due to the underlying pigmented epithelium of the ciliary body and pars plana, for example, when greater than 90% of the sclera in a furrow has been ablated, the surgeon can reduce the power setting of the device 60 or can otherwise stop using the device to avoid penetrating the eyeball. For example, with the exemplary device disclosed above (i.e., the “Fugo Blade®”) there are two adjustable control knobs (not shown). The first control can be set in one of three settings, namely, low, medium or high. The second control knob has settings from 1 to 10 in integers. For creating a furrow of approximately 600 microns, the first control is set to medium and the second set at “7.”

It should be pointed out at this juncture that the size of the electrode tip 80 need not be of a diameter which is the width of the furrow(s) to be produced. In this regards, when creating a furrow of approximately 600 microns, a tip having a diameter of considerably less than 600 microns can be used because the energy spreads about 25-50 microns beyond the tip. Thus, for a range furrow widths of 200-2000 microns, with a depth of at least 90% of the thickness of the sclera the tips can be approximately 50-700 microns in diameter. Moreover, the tips need not be circular in shape.

While the invention has been described in detail and with reference to specific examples thereof, it will be apparent to one skilled in the art that various changes and modifications can be made therein without departing from the spirit and scope thereof.

I claim:

1. An ophthalmic surgical method for treating a living being having presbyopia comprising applying radio frequency energy to the sclera of the eye in a desired pattern to remove portions of the scleral tissue in said pattern, said pattern comprising at least one furrow, said at least one furrow having a width in the range of approximately 200 to approximately 2000 microns and a depth in the range of approximately ninety percent to slightly less than one hundred percent of the thickness of the sclera.

2. The ophthalmic surgical method of claim 1 wherein said pattern comprises plural furrows.

3. The ophthalmic surgical method of claim 2 wherein each of said plural furrows are of the same shape.

4. The ophthalmic surgical method of claim 3 wherein each of said plural furrows are of the same size.

5. The ophthalmic surgical method of claim 1 wherein said at least one furrow is in the range of approximately 600 to approximately 1000 microns in width.

6. The ophthalmic surgical method of claim 1 wherein said at least one furrow is in the range of approximately ninety percent to approximately ninety-nine percent of the thickness of the sclera.

7. The ophthalmic surgical method of claim 5 wherein said at least one furrow is in the range of approximately ninety percent to approximately ninety-nine percent of the thickness of the sclera.

8. The ophthalmic surgical method of claim 2 wherein at least one of said plural furrows is in the range of approximately 600 to approximately 1000 microns in width.

9. The ophthalmic surgical method of claim 2 wherein at least one of said plural furrows is in the range of approximately ninety percent to approximately ninety-nine percent of the thickness of the sclera.

10. The ophthalmic surgical method of claim 8 wherein said at least one of said plural furrow is in the range of approximately ninety percent to approximately ninety-nine percent of the thickness of the sclera.

11. The ophthalmic surgical method of claim 1 wherein said at least one furrows is of a length in the range of approximately 3 to 7 millimeters.

12. The ophthalmic surgical method of claim 11 wherein said at least one furrow is of a length in the range of approximately 4.5 millimeters.

13. The ophthalmic surgical method of claim 2 wherein at least one of said plural furrows is of a length in the range of approximately 3 to 7 millimeters.

14. The ophthalmic surgical method of claim 13 wherein said at least one of said plural furrows is of a length in the range of approximately 4.5 millimeters.

15. The ophthalmic surgical method of claim 1 wherein said at least one furrow is generally linear in shape.

16. The ophthalmic surgical method of claim 1 wherein said at least one furrow includes at least a portion that is arcuate in shape.

17. The ophthalmic surgical method of claim 2 wherein at least one of said plural furrows is generally linear in shape.

18. The ophthalmic surgical method of claim 2 wherein at least one of said plural furrow includes at least a portion that is arcuate in shape.

19. The ophthalmic surgical method of claim 2 wherein said desired pattern comprises an array of generally radially extending furrows, each of which having an end portion closely adjacent the limbus.

20. The ophthalmic surgical method of claim 2 wherein said desired pattern comprises an array of generally radially extending furrows, at least a respective of said furrows extending into a respective quadrant of the sclera.

21. The ophthalmic surgical method of claim 19 wherein said pattern comprises at least four furrows.

22. The ophthalmic surgical method of claim 21 wherein said furrows are equidistantly disposed about the limbus.

23. The ophthalmic surgical method of claim 1 wherein said method comprises providing a radio frequency device having an electrode tip arranged for application to the sclera of the eye and another electrode for engagement with a portion of the body of the living being and wherein said device is operated by bringing said tip adjacent the scleral tissue of the eye so that said electrode tip removes a portion of the scleral tissue in said desired pattern.

24. The ophthalmic surgical method of claim 1 comprising placing visible indicia on the sclera to serve as a guide for forming said at least one furrow.

25. The ophthalmic surgical method of claim 2 comprising placing visible indicia on the sclera to serve as a guide for forming said at least one furrow.

26. The ophthalmic surgical method of claim 24 comprising utilizing a template for placing said visible indicia on the sclera.

27. The ophthalmic surgical method of claim 25 comprising utilizing a template for placing said visible indicia on the sclera.

28. A system for surgically treating presbyopia in a living being, said system comprising a template and a radio frequency energy applying device, said template being arranged for application to the eye of the being to enable at least one mark to be placed on the eye to indicate the location of a furrow to be created in the sclera of the eye, said radio-frequency energy applying device comprising an electrode member adapted to be positioned adjacent the mark on the eye to direct radio frequency energy adjacent or at the mark to ablate the underlying sclera and thereby create a furrow having a width in the range of approximately 200 to approximately 2000 microns and a depth in the range of approximately ninety percent to slightly less than one hundred percent of the thickness of the sclera.

29. The system of claim 28 wherein said device comprises a low power radio-frequency energy generator, an ablating electrode and a grounding electrode, said grounding electrode of one polarity electrically coupled to said generator and being arranged for engagement with a portion of the body of the being, said ablating electrode having a small tip of another polarity electrically coupled to said generator, said tip being arranged to be positioned at or closely adjacent the mark.

30. The system of claim 29 wherein the amount of radio-frequency energy produced by said generator is adjustable.
